SEQUENCE LISTING (4 sequences)

SEQ ID NO: 1 - DNA, 1994 bp, Homo sapiens
  CDS (149)..(1405)
ggcacgagcatggcccttgtgatccaggtggggaaactaaggcccagagaagtgaggaccccgcagactatcaatcccagtctcttcccctcactccctgtgaagctctccagcatcatcgaggtcccatcagcccttgccctgttggatgaataggcacctctggaagagccaactgtgtgagatggtgcagcccagtggtggcccggcagcagatcaggacgtactgggcgaagagtctcctctggggaagccagccatgctgcacctgccttcagaacagggcgctcctgagaccctccagcgctgcctggaggagaatcaagagctccgagatgccatccggcagagcaaccagattctgcgggagcgctgcgaggagcttctgcatttccaagccagccagagggaggagaaggagttcctcatgtgcaagttccaggaggccaggaaactggtggagagactcggcctggagaagctcgatctgaagaggcagaaggagcaggctctgcgggaggtggagcacctgaagagatgccagcagcagatggctgaggacaaggcctctgtgaaagcccaggtgacgtccttgctcggggagctgcaggagagccagagtcgcttggaggctgccactaaggaatgccaggctctggagggtcgggcccgggcggccagcgagcaggcgcggcagctggagagtgagcgcgaggcgctgcagcagcagcacagcgtgcaggtggaccagctgcgcatgcagggccagagcgtggaggccgcgctccgcatggagcgccaggccgcctcggaggagaagaggaagctggcccagttgcaggtggcctatcaccagctcttccaagaatacgacaaccacatcaagagcagcgtggtgggcagtgagcggaagcgaggaatgcagctggaagatctcaaacagcagctccagcaggccgaggaggccctggtggccaaacaggaggtgatcgataagctgaaggaggaggccgagcagcacaagattgtgatggagaccgttccggtgctgaaggcccaggcggatatctacaaggcggacttccaggctgagaggcaggcccgggagaagctggccgagaagaaggagctcctgcaggagcagctggagcagctgcagagggagtacagcaaactgaaggccagctgtcaggagtcggccaggatcgaggacatgaggaagcggcatgtcgaggtctcccaggcccccttgccccccgcccctgcctacctctcctctcccctggccctgcccagccagaggaggagcccccccgaggagccacctgacttctgctgtcccaagtgccagtatcaggcccctgatatggacaccctgcagatacatgtcatggagtgcattgagtagggccggccagtgcaaggccactgcctgcccgaggacgtgcccgggaccgtgcagtctgcgctttcctctcccgcctgcctagcccaggatgaagggctgggtggccacaactgggatgccacctggagccccacccaggagctggccgcggcaccttacgcttcagctgttgatccgctggtcccctcttttggggtagatgcggccccgatcaggcctgactcgctgctctttttgttcccttctgtctgctcgaaccacttgcctcgggctaatccctccctcttcctccacccggcactggggaagtcaagaatggggcctggggctctcagggagaactgcttcccctggcagagctgggtggcagctcttcctcccaccggacaccgacccgcccgccgctgtgccctgggagtgctgccctcttaccatgcacacgggtgctctccttttgggctgcatgctattccattttgcagccagaccgatgtgtatttaaccagtcactattgatggacatttgggttgtttcccatctttttgttaccataaataatggcatagtaaaaaaaaaaaaaaaaaa

SEQ ID NO: 2 - protein, 419 aa, Homo sapiens
MNRHLWKSQLCEMVQPSGGPAADQDVLGEESPLGKPAMLHLPSEQGAPETLQRCLEENQELRDAIRQSNQILRERCEELLHFQASQREEKEFLMCKFQEARKLVERLGLEKLDLKRQKEQALREVEHLKRCQQQMAEDKASVKAQVTSLLGELQESQSRLEAATKECQALEGRARAASEQARQLESEREALQQQHSVQVDQLRMQGQSVEAALRMERQAASEEKRKLAQLQVAYHQLFQEYDNHIKSSVVGSERKRGMQLEDLKQQLQQAEEALVAKQEVIDKLKEEAEQHKIVMETVPVLKAQADIYKADFQAERQAREKLAEKKELLQEQLEQLQREYSKLKASCQESARIEDMRKRHVEVSQAPLPPAPAYLSSPLALPSQRRSPPEEPPDFCCPKCQYQAPDMDTLQIHVMECIE

SEQ ID NO: 3 - DNA, 5371 bp, Homo sapiens
  CDS (392)..(3262)
gggggcgggcccaggtagcaggtttggctgcgcgggggccgcgcgtcggagtttccccctttctagggtgaggatggttctacacagccacccggagttccttagttgaaaggtgcgccctgctgtgacagaatgtggtaattgtaatctttaacattttcatgtaaaacatatttcctgatcatctttccattgtcttcatggaaaattgataaatatttgtgccttccaactctcgtcttggttgaatgacttcatcttaatacaacatggacaccacgttgctgaaaacatgctttgggactgccactgaatttatcttttgcggttttatgacaaagttattagtagtttcccttttttgaattagtattttgaagttaatatcacaatgagttcaggcttatggagccaagaaaaagtcacttcaccctactgggaagagcggattttttacttgcttcttcaagaatgcagcgttacagacaaacaaacacaaaagctccttaaagtaccgaagggaagtataggacagtatattcaagatcgttctgtggggcattcaaggattccttctgcaaaaggcaagaaaaatcagattggattaaaaattctagagcaacctcatgcagttctctttgttgatgaaaaggatgttgtagagataaatgaaaagttcacagagttacttttggcaattaccaattgtgaggagaggttcagcctgtttaaaaacagaaacagactaagtaaaggcctccaaatagacgtgggctgtcctgtgaaagtacagctgagatctggggaagaaaaatttcctggagttgtacgcttcagaggacccctgttagcagagaggacagtctccggaatattctttggagttgaattgctggaagaaggtcgtggtcaaggtttcactgacggggtgtaccaagggaaacagctttttcagtgtgatgaagattgtggcgtgtttgttgcattggacaagctagaactcatagaagatgatgacactgcattggaaagtgattacgcaggtcctggggacacaatgcaggtcgaacttcctcctttggaaataaactccagagtttctttgaaggttggagaaacaatagaatctggaacagttatattctgtgatgttttgccaggaaaagaaagcttaggatattttgttggtgtggacatggataaccctattggcaactgggatggaagatttgatggagtgcagctttgtagttttgcgtgtgttgaaagtacaattctattgcacatcaatgatatcatcccagctttatcagagagtgtgacgcaggaaaggaggcctcccaaacttgcctttatgtcaagaggtgttggggacaaaggttcatccagtcataataaaccaaaggctacaggatctacctcagaccctggaaatagaaacagatctgaattattttataccttaaatgggtcttctgttgactcacaaccacaatccaaatcaaaaaatacatggtacattgatgaagttgcagaagaccctgcaaaatctcttacagagatatctacagactttgaccgttcttcaccaccactccagcctcctcctgtgaactcactgaccaccgagaacagattccactctttaccattcagtctcaccaagatgcccaataccaatggaagtattggccacagtccactttctctgtcagcccagtctgtaatggaagagctaaacactgcacccgtccaagagagtccacccttggccatgcctcctgggaactcacatggtctagaagtgggctcattggctgaagttaaggagaaccctcctttctatggggtaatccgttggatcggtcagccaccaggactgaatgaagtgctcgctggactggaactggaagatgagtgtgcaggctgtacggatggaaccttcagaggcactcggtatttcacctgtgccctgaagaaggcgctgtttgtgaaactgaagagctgcaggcctgactctaggtttgcatcattgcagccggtttccaatcagattgagcgctgtaactctttagcatttggaggctacttaagtgaagtagtagaagaaaatactccaccaaaaatggaaaaagaaggcttggagataatgattgggaagaagaaaggcatccagggtcattacaattcttgttacttagactcaaccttattctgcttatttgcttttagttctgttctggacactgtgttacttagacccaaagaaaagaacgatgtagaatattatagtgaaacccaagagctactgaggacagaaattgttaatcctctgagaatatatggatatgtgtgtgccacaaaaattatgaaactgaggaaaatacttgaaaaggtggaggctgcatcaggatttacctctgaagaaaaagatcctgaggaattcttgaatattctgtttcatcatattttaagggtagaacctttgctaaaaataagatcagcaggtcaaaaggtacaagattgttacttctatcaaatttttatggaaaaaaatgagaaagttggcgttcccacaattcagcagttgttagaatggtcttttatcaacagtaacctgaaatttgcagaggcaccatcatgtctgattattcagatgcctcgatttggaaaagactttaaactatttaaaaaaatttttccttctctggaattaaatataacagatttacttgaagacactcccagacagtgccggatatgtggagggcttgcaatgtatgagtgtagagaatgctacgacgatccggacatctcagctggaaaaatcaagcagttttgtaaaacctgcaacactcaagtccaccttcatccgaagaggctgaatcataaatataacccagtgtcacttcccaaagacttacccgactgggactggagacacggctgcatcccttgccagaatatggagttatttgctgttctctgcatagaaacaagccactatgttgcttttgtgaagtatgggaaggacgattctgcctggctcttctttgacagcatggccgatcgggatggtggtcagaatggcttcaacattcctcaagtcaccccatgcccagaagtaggagagtacttgaagatgtctctggaagacctgcattccttggactccaggagaatccaaggctgtgcacgaagactgctttgtgatgcatatatgtgcatgtaccagagtccaacaatgagtttgtacaaataactggggtcatcgggaaaggcaaagaaactgaaggcagagtcctaacgttgcatcttattcgagctggcagttctgttcacgtccattgccggcaatggatgtctttgtggtgatgatccttcagaaaaggatgcctctgtttaaaaacaaattgcttttgtgtccctgaagtatttaataagaagcattttgcactctagaaagtatgtttgtgttggttttttaagaagtctaaatgaagttattaatacctgaagctttaagttaagtgcattgatcatatgatatttttggaagcatacaattttaattgtggaagtttaaagcctcttttagtccattgagaatgtaaataaatgtgtcttctttatggaccaaggatatgaaatcatttttcttttgtagctaacggttgccttgaggaagaaataatttggttttattaagagtctactctcaatccagttattagagatgtactgagtttgatttgttaatcctttctatatactgctgatcttgcatgtctacaatctgctcagtttttctgtgtttctgcaatagtggtcagaaaaatacttaaattcccttaatggtgttgttttctatttgttctggttttgagataaatgagtgattctgtccccaaatgtccatttttgaagtgattttcctggaggattagggtatttagcagttgaagctcttcattcatagtagttactgtcagctaacaggttttttaaggcttttaactattaatattttatggaatggggcaaagtaaattgatgaaagaattggagtgataatagtcctttacaaacatacagtccataagaaaatgaatttggcatatagaattattacaatttcctgggagagatggatatttaaacctctattattttagacaagactgtctagaacttaagtttgatctgtcagccagtactcccattaaattcagtgtagtttcacttgatagaatcagatatgttatcgaaatgttagcagcagcttcatcctccttctgattaaagtaagtagaaatgggatgttttgtttaataacagccatagtgtgtgtttagaccacagcggatgttgtagaccaggaccatagatgatacatgtcagtgctgtggaatgtgcattctctgagtgttgttttgtggtatcattgtctttcctgaatgactttctaactgtgcagaaaggcagaaaagtcatcatatgtatatgtcatatgactttataaaatatttaatgtgacaaaaagtggaaagaatctttacaaaccctgcaattacttttttaaaggcacttttactctttggttttatcattccattttgctaatatttactagctttataaattacagtaaggtacaaaaactcatcttgtaatattttcatttttgaagtgaaaaagtacatatattttgcacaaggttttatactgctaagtgcttggttggggtggtgagatgatgattagatcaggggtgaggctgagagactctgggtttagggctagccctgcctccatctcccttgggtaaaatgaagggtgtggggtaaaagatgcataaggccttttctagctctgacagcctagaagtccaatcaccctgtaataaatatgtgttgaatgaagaaatgggtgaatgagcttgtcaatgtgattttaaaaaattgactacctggaggaatgattaggaatctaaatgaagccagccctcggtatctgcaggtttctcatccatggattcaaccaactgcaaatggaaaatacgattttttttaaaaaaaggatggttacatccgtattgaacatgtacagacttttttcttgtcattattctctgaacaatacaagaactctttatgtagcatttacatttattaggtattataagtaatctagagattatttaattaaaatatacaggaggatgtgtgtttatatgccagaaattctgtaccattttgtatcagggaattgagcatcttcagatgttggtatctgcagggatcctggaaccaaacccctgcagatactaagggctgacgatctaggtaagactggatttaacagttggaaa

SEQ ID NO: 4 - protein, 956 aa, Homo sapiens
MSSGLWSQEKVTSPYWEERIFYLLLQECSVTDKQTQKLLKVPKGSIGQYIQDRSVGHSRIPSAKGKKNQIGLKILEQPHAVLFVDEKDVVEINEKFTELLLAITNCEERFSLFKNRNRLSKGLQIDVGCPVKVQLRSGEEKFPGVVRFRGPLLAERTVSGIFFGVELLEEGRGQGFTDGVYQGKQLFQCDEDCGVFVALDKLELIEDDDTALESDYAGPGDTMQVELPPLEINSRVSLKVGETIESGTVIFCDVLPGKESLGYFVGVDMDNPIGNWDGRFDGVQLCSFACVESTILLHINDIIPALSESVTQERRPPKLAFMSRGVGDKGSSSHNKPKATGSTSDPGNRNRSELFYTLNGSSVDSQPQSKSKNTWYIDEVAEDPAKSLTEISTDFDRSSPPLQPPPVNSLTTENRFHSLPFSLTKMPNTNGSIGHSPLSLSAQSVMEELNTAPVQESPPLAMPPGNSHGLEVGSLAEVKENPPFYGVIRWIGQPPGLNEVLAGLELEDECAGCTDGTFRGTRYFTCALKKALFVKLKSCRPDSRFASLQPVSNQIERCNSLAFGGYLSEVVEENTPPKMEKEGLEIMIGKKKGIQGHYNSCYLDSTLFCLFAFSSVLDTVLLRPKEKNDVEYYSETQELLRTEIVNPLRIYGYVCATKIMKLRKILEKVEAASGFTSEEKDPEEFLNILFHHILRVEPLLKIRSAGQKVQDCYFYQIFMEKNEKVGVPTIQQLLEWSFINSNLKFAEAPSCLIIQMPRFGKDFKLFKKIFPSLELNITDLLEDTPRQCRICGGLAMYECRECYDDPDISAGKIKQFCKTCNTQVHLHPKRLNHKYNPVSLPKDLPDWDWRHGCIPCQNMELFAVLCIETSHYVAFVKYGKDDSAWLFFDSMADRDGGQNGFNIPQVTPCPEVGEYLKMSLEDLHSLDSRRIQGCARRLLCDAYMCMYQSPTMSLYK

(19) United States
(12) Patent Application Publication
(10) Pub. No.: US 2005/0131648 A1
(43) Pub. Date: Jun. 16, 2005

(54) Screening assays for agonists or antagonists of CD40 signaling

(75) Inventors: Jonathan M. J. Derry, Bainbridge Island, WA (US); William C. Fanslow III, Normandy Park, WA (US); William C. Dougall, Seattle, WA (US)

Correspondence Address: Immunex Corporation/PAP;Law Department, 51 University Street, Seattle, WA, 98101, US

(73) Assignee: Immunex Corporation

(21) Appl. No.: 10/760,678

(22) Filed: Jan. 20, 2004

Related U.S. Application Data
(63) Continuation of application No. 09/851,673, filed on May 8, 2001, now abandoned.

(51) Int. Cl. G01N 33/53; G06F 19/00; G01N 33/48; G01N 33/50

(52) U.S. Cl. 702/19; 435/7.1

(57) ABSTRACT

The present invention provides methods for identifying a molecule that antagonizes or agonizes CD40 activity by screening for molecules that modulate the binding of NEMO and CYLD. Also provided are assays useful in screening for the binding of NEMO and CYLD and databases and methods useful for analyzing the resultant information.

### CROSS REFERENCE TO RELATED APPLICATIONS

This application is a continuation of application U.S. Ser. No. 09/851,673, filed May 8, 2001, which is incorporated by reference herein.

### FIELD OF THE INVENTION

This invention relates to methods for screening for agonists and/or antagonists of activities associated with TNF receptor family member CD40.

### BACKGROUND OF THE INVENTION

CD40 is a member of the tumor necrosis factor (TNF)/nerve growth factor (NGF) receptor family, which is defined by the presence of cysteine-rich motifs in the extracellular region (Smith et al., Science 248:1019, 1990; Mallett and Barclay, Immunology Today 12:220, 1991; Locksley et al., Cell 104:487, 2001). This family includes the lymphocyte antigen CD27, CD30 (an antigen found on Hodgkin's lymphoma and Reed-Sternberg cells), two receptors for TNF, a murine protein referred to as 4-1BB, rat OX40 antigen, NGF receptor, and Fas antigen. Similar to other members of the TNF/NGF receptor family, CD40 contains a leader sequence, trans-membrane domain an extracellular domain responsible for binding its native cognate, CD40 ligand (CD40L; described in U.S. Pat. Nos. 5,961,974, 5,962,406 and 5,981,724; hereinafter, the Armitage patents). CD40 has been found to be expressed on B lymphocytes, monocytes/macrophages, dendritic cells, smooth muscle cells, microglia, epithelial cells and some carcinoma cell lines.

The cytoplasmic domain of CD40 associates intracellularly with several of the TNF receptor-associated factors (TRAFs; Baker and Reddy, Oncogene 12:1 (1996)) including TRAFs 2, 3, 5 and 6 (Pullen et al., Biochemistry 37:11836, 1998; Cao et al., Nature 383:443, 1996; Ishida et al., Proc. Natl. Acad. Sci. USA 93:9437, 1996). The TRAFs are cytoplasmic proteins that often mediate signal transduction by members of the TNF receptor superfamily, and they are important in the regulation of, for example, immune and inflammatory responses.

Triggering of CD40, such as by contact with membrane-bound or soluble CD40 ligand (CD40L), results in the stimulation of CD40-mediated cellular responses. These cellular responses can include the activation of transcription factor NF-kappaB, a ubiquitous transcription factor that is extensively utilized in cells of the immune system. NF-kappaB is normally maintained in the cytoplasm by interaction with IkappaB members (reviewed in Karin, Oncogene 18: 6867, 1999; Chen and Gosh, Oncogene 18:6845, 1999). Release of NF-kappaB, and subsequent translocation of this transcription factor to the nucleus, result from phosphorylation of IkappaB, which leads to its ubiquitinization and degradation. Phosphorylation of IkappaB is mediated by the inducible kappaB kinase (IKK) complex consisting of two kinases, IKKalpha, and IKKbeta, and a scaffolding protein referred to as NEMO (NF-kappaB essential modulator; Yamaoka et al., Cell 93:1231, 1998), IKK-gamma (Rothwarf et al., Nature 395:297, 1998), FIP3 (Fourteen K interacting protein 3; Li et al., Proc. Natl. Acad. Sci. USA 96:1042, 1999) or RAP2 (RIP-associated protein 2; WO 99/47672) (hereinafter, NEMO).

NEMO exhibits characteristics of a “signaling scaffold protein”, and binds not only to kinases involved in NF-kappaB-mediated signaling, but to proteins that play a role in signaling via TNF and/or Fas (Receptor Interacting Protein 2 or RIP2, McCarty et al., J. Biol. Chem. 273:16968, 1998; NF-kappaB Inducing Kinase, NIK, Malinin et al., Nature 385:640, 1997; and A20, Cooper et al., J. Biol. Chem. 271:18068, 1996), to several viral proteins (Adeno 14.7K, Li et al., supra; HTLV Tax, Yamaoka et al., supra), and to proteins of unknown function (TIP60, Tat interactive protein 60 kDa, Kamine et al., Virology 216:357, 1996; CYLD, a putative tumor suppressor gene associated with familial cylindromatosis, Bignell et al., Nat. Genet. 25:160, 2000).

Additionally, a peptide that blocks the interaction of NEMO with IKK-alpha/IKK-beta has been shown to be potent anti-inflammatory agent (May et al., Science 289:1550, 2000). Moreover, deletion mutations of NEMO have been shown to cause familial incontinentia pigmentosa (IP; Smahi et al., Nature 405:466, 2000), whereas milder mutations in the carboxy-terminal region of NEMO have been linked to ectodermal dysplasia with immunodeficiency/hyperIgM (BED-ID; Zonano et al., Am. J. Hum. Genet. 67:1555, 2000; Jain et al., Nature Immunology 2:223, 2001). Because of the role NEMO plays in numerous signaling pathways, there is a need in the art to identify molecules that can affect the interaction of NEMO with certain molecules (either as antagonists or agonists), to allow regulation of specific pathways in an inflammatory response.

### SUMMARY OF THE INVENTION

The present invention provides methods for screening for a molecule that antagonizes or agonizes the activity of NEMO in CD40 signaling. In one aspect of the invention, there is provided a method for identifying compounds that alter CD40 signaling activity comprising: (a) mixing a test compound with a polypeptide selected from the group consisting of (i) a NEMO polypeptide comprising amino acids 287 through 419 SEQ ID NO:2, (ii) a fragment of a NEMO polypeptide according to (i) that is capable of binding a CYLD polypeptide according to SEQ ID NO:4 or fragment or variant thereof, and (iii) variants of the NEMO polypeptides of (i) and (ii); and (b) determining whether the test compound alters the ability of NEMO to bind CYLD. The invention further provides a method for identifying compounds that inhibit binding of NEMO and CYLD comprising: (a) mixing a test compound with a polypeptide selected from the group consisting of (i) a NEMO polypeptide comprising amino acids 287 through 419 SEQ ID NO:2, (ii) a fragment of a NEMO polypeptide according to (i) that is capable of binding a CYLD polypeptide according to SEQ ID NO:4, and (iii) variants of the NEMO polypeptides of (i) and (ii), and a binding partner of said NEMO polypeptide selected from the group consisting of (iv) a CYLD polypeptide according to SEQ ID NO:4, (v) a fragment of a CYLD polypeptide according to SEQ ID NO:4 that is capable of binding a NEMO polypeptide of (i), (ii), or (iii), and (vi) variants of the CYLD polypeptides of (iv) and (v); and (b) determining whether the test compound inhibits the binding activity of said NEMO and CYLD polypeptides.

In one aspect, the inventive methods utilize homogeneous assay formats such as fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence. In another aspect, the inventive methods utilize heterogeneous assay formats such as enzyme-linked immunosorbant assays (ELISA) or radioimmunoassays. In yet another aspect of the invention are cell-based assays, for example those utilizing reporter genes, as well as functional assays that analyze the effect of an antagonist or agonist on biological function(s).

The invention further provides methods for producing information comprising the identity of a compound that alters one or more biological activities of CD40, the method comprising using assay methods of the invention to identify one or more compounds that alter the binding of NEMO and CYLD. In one preferred embodiment, the compound decreases (or antagonizes) the binding of NEMO and CYLD, and in another distinct embodiment, the compound increases (or agonizes) the binding of NEMO and CYLD.

Preferably the biological activity of CD40 that is decreased or downregulated is selected from the group consisting of deleterious effects of CD40-mediated immune or inflammatory response (including atherosclerosis, arthritis, multiple sclerosis (MS), systemic lupus erythematosous (SLE), thrombosis, graft versus host disease and/or graft rejection. In a distinct embodiment, the biological activity of CD40 that is increased or upregulated is selected from the group consisting of upregulation of a cell-mediated immune response, upregulation of an antibody-mediated immune response, prevention or treatment of infectious disease, and prevention or treatment of neoplastic disease.

Also provided by the invention is the information produced according to the inventive methods, said information comprising the identity of a compound that alters the biological activity of CD40, and preferably embodied in a storage medium selected from the group consisting of the brains of living organisms, paper, magnetic tape, optical tape, floppy disks, compact disks, computer system hard drives, and computer memory units. In a further aspect, the invention provides a database comprising said information, wherein the information is preferably embodied in a computer-readable medium, and a separate embodiment wherein the information is embodied in a human-readable medium.

Additionally provided by the invention is a computer system comprising a database containing records pertaining to a plurality of compounds, wherein the records comprise results of an assay of the invention, and a user interface allowing a user to access information regarding the plurality of compounds. In another aspect of the invention, a computer system is provided for storing and retrieving data on a plurality of compounds, the computer system comprising: (a) input means for entering data for the compounds into a storage medium; (b) a processor for creating an individual record for each compound, the processor assigning specific identifying values for each compound; (c) means for selecting one or more of the records based on results in an assay; and (d) means for transmitting information in the record or records to an output device to produce a report; preferably a report in human-readable form, and wherein the computer system preferably further comprises a video display unit.

The invention also provides a method of using the computer system of the invention to select one or more compounds for testing from a plurality of compounds having records stored in a database, the method comprising: displaying a list of said records or a field for entering information identifying one or more of said records; and
selecting one or more of the records from the list or the record or records identified by entering information in the field. Further, the invention provides a method of operating a computer system for analyzing compounds that modulate the interaction NEMO and CYLD, the method comprising: (a) entering data relating to a plurality of compounds into a storage medium; (b) processing the data to create an individual record for each compound; (c) testing compounds for the ability to modulate binding of NEMO to CYLD; and (d) communicating results from the testing into the storage medium such that results for each compound are associated with the individual record for that compound; wherein in one embodiment the storage medium comprises one or more computer memory units, and in another embodiment the computer system further comprises a video display unit.

In yet another aspect of the invention, a database is provided comprising records generated according to the methods of the invention, and a method is provided for selecting compounds that modulate the interaction of NEMO and CYLD, comprising compiling said database, analyzing the testing results, and selecting one or more compounds.

Candidate molecules that are determined to agonize or antagonize a CD40 signaling activity of NEMO are useful, for example, for the further definition of CD40-mediated signaling pathways, and for the manipulation of CD40-mediated cellular responses. Moreover, CD40 signaling agonists and antagonists provide therapeutic agents for treating disorders of the immune system, and inflammatory disorders, as well as treatment of conditions characterized by malignant cells expressing CD40.

### BRIEF DESCRIPTION OF THE DRAWINGS

FIG. 1 depicts the structure NEMO, with various regions highlighted; the amino acid sequence is shown in SEQ ID NO:2. The region from amino acid 217 through 419 is believed to bind CYLD; homodimerization is thought to be mediated by amino acids 217 through 264. Binding to NIK and TIP60 is believed to occur between amin acids 95 and 264, while binding to RIP and A20 is through amino acids 95 through 218. NEMO binds to 14.7K via amino acids 180 through 419; binding to IKK-1 and IKK-2 is mediated by amino acids 44 through 86. The majority of mutations that result in familial IP are deletions of exons 3 through 10. The stippled regions labeled ‘αH’ are alpha-helical regions ‘LZ’ represents the leucine zipper region, and ‘ZF’ denotes the zinc finger. Amino acids 397, 400, 413 and 417 are believed to coordinate zinc.

### DETAILED DESCRIPTION OF THE PREFERRED EMBODIMENT

The present invention provides methods for screening for a molecule that antagonizes or agonizes CD40 signaling activity, utilizing molecules that play a role in signaling through CD40. Two patients with a form of X-linked hyper IgM syndrome with ectodermal dysplasia and normal CD40 and CD40L expression were found to have mutations in NEMO. In one, a T to C mutation at nucleotide 1249 results in a Cys to Arg substitution at amino acid 417 of NEMO (C417R), and in the other, an A to T substitution at nucleotide 1217 results in an Asp to Val mutation at amino acid 406 (V406D). B cells from these patients did not undergo immunoglobulin class switching when contacted with a soluble, oligomeric form of CD40L (described in the Armitage patents, supra), and also failed to upregulate CD54 expression. Moreover, antigen-presenting cells from these patient did not synthesize Interleukin-12 (IL-12) and TNF-alpha when stimulated with CD40L and interferon-gamma (IFN-gamma), but did synthesize these two cytokines when stimulated with S. aureus Cowan protein A plus IFN-gamma or lipopolysaccharide plus IFN-gamma. Additionally, degradation of IkappaB-alpha in response to TNF or LPS appeared normal in monocytes from these patients, but stimulation with CD40L failed to induce this response.

These findings demonstrated that the zinc finger region of NEMO represents an area of NEMO that is critical for CD40 signal transduction, but that does not appear critical for transduction of signal via other pathways leading to NF-kappaB activation (for example, binding of LPS through the Toll-like receptors, and/or binding of TNF to TNF receptor). Accordingly, peptides derived from this region are likely to be useful in screening for small molecules that inhibit the interaction of this domain of NEMO with other molecules in the CD40 signaling cascade (i.e., CYLD), which will be useful in downregulating or controlling deleterious effects of CD40-mediated immune or inflammatory response. Conditions that are thought to be mediated by CD40 signaling include atherosclerosis, arthritis, multiple sclerosis (MS), systemic lupus erythematosous (SLE), thrombosis, graft versus host disease and/or graft rejection.

Candidate Molecules to be Tested for CD40 Signaling Activity:

The methods of the invention may be used to identify antagonists and agonists of CD40 signaling activity from cells, cell-free preparations, chemical libraries, cDNA libraries, recombinant antibody libraries (or libraries comprising subunits of antibodies) and natural product mixtures. The antagonists and agonists may be natural or modified substrates, ligands, enzymes, receptors, etc. of the polypeptides of the instant invention, or may be structural or functional mimetics of one of the polypeptides (NEMO or CYLD). Potential antagonists of the instant invention may include small molecules, peptides and antibodies that bind to and occupy a binding site of the inventive polypeptides or a binding partner thereof, causing them to be unavailable to bind to their natural binding partners and therefore preventing normal biological activity. Potential agonists include small molecules, peptides and antibodies which bind to the instant polypeptides or binding partners thereof, and elicit the same or enhanced biologic effects as those caused by the binding of the polypeptides of the instant invention.

Small molecule agonists and antagonists are usually less than 10K molecular weight and may possess a number of physicochemical and pharmacological properties which enhance cell penetration, resist degradation and prolong their physiological half-lives (Gibbs, J., Pharmaceutical Research in Molecular Oncology, Cell, Vol. 79 (1994)). Antibodies, which include intact molecules as well as fragments such as Fab and F(ab′)2 fragments, as well as recombinant molecules derived therefrom (including antibodies expressed on phage, intrabodies, single chain antibodies such as scFv and other molecules derived from immunoglobulins that are known in the art), may be used to bind to and inhibit the polypeptides of the instant invention by blocking the propagation of a signaling cascade. It is preferable that the antibodies are humanized, and more preferable that the antibodies are human. The antibodies of the present invention may be prepared by any of a variety of well-known methods.

Additional examples of candidate molecules, also referred to herein as “test molecules,” to be tested for CD40 signaling agonist or antagonist activity include, but are not limited to, carbohydrates, small molecules (usually organic molecules or peptides), proteins, and nucleic acid molecules (including oligonucleotide fragments typically consisting of from 8 to 30 nucleic acid residues). Peptides to be tested typically consist of from 5 to 25 amino acid residues. Also, candidate nucleic acid molecules can be antisense nucleic acid sequences, and/or can possess ribozyme activity. Small molecules to be screened using the hereindescribed screening assays can typically be administered orally or by injection to a patient in need thereof. Small molecules that can be administered orally are especially preferred. The small molecules of the invention preferably will not be toxic at the doses required for them to be effective as pharmaceutical agents, and they are preferably not subject to rapid loss of activity in the body, such as the loss of activity that might result from rapid enzymatic or chemical degradation. In addition, pharmaceutically useful small molecules are preferably not immunogenic.

The methods of the invention can be used to screen for antisense molecules that inhibit the functional expression of one or more mRNA molecules that encode one or more proteins that mediate a CD40-dependent cellular response. An anti-sense nucleic acid molecule is a DNA sequence that is inverted relative to its normal orientation for transcription and so expresses an RNA transcript that is complementary to a target mRNA molecule expressed within the host cell (i.e., the RNA transcript of the anti-sense nucleic acid molecule can hybridize to the target mRNA molecule through Watson-Crick base pairing). An anti-sense nucleic acid molecule may be constructed in a number of different ways provided that it is capable of interfering with the expression of a target protein. Typical anti-sense oligonucleotides to be screened preferably are 30-40 nucleotides in length. The anti-sense nucleic acid molecule generally will be substantially identical (although in antisense orientation) to the target gene. The minimal identity will typically be greater than about 65%, but a higher identity might exert a more effective repression of expression of the endogenous sequences. Substantially greater identity of more than about 80% is preferred, though about 95% to absolute identity would be most preferred.

Candidate nucleic acid molecules can possess ribozyme activity. Thus, the methods of the invention can be used to screen for ribozyme molecules that inhibit the functional expression of one or more mRNA molecules that encode one or more proteins that mediate a CD40 dependent cellular response. Ribozymes are catalytic RNA molecules that can cleave nucleic acid molecules having a sequence that is completely or partially homologous to the sequence of the ribozyme. It is possible to design ribozyme transgenes that encode RNA ribozymes that specifically pair with a target RNA and cleave the phosphodiester backbone at a specific location, thereby functionally inactivating the target RNA. In carrying out this cleavage, the ribozyme is not itself altered, and is thus capable of recycling and cleaving other molecules. The inclusion of ribozyme sequences within antisense RNAs confers RNA-cleaving activity upon them, thereby increasing the activity of the antisense constructs.

The design and use of target RNA-specific ribozymes is described in Haseloff et al. (Nature, 334:585, 1988; see also U.S. Pat. No. 5,646,023), both of which publications are incorporated herein by reference. Tabler et al. (Gene 108:175, 1991) have greatly simplified the construction of catalytic RNAs by combining the advantages of the anti-sense RNA and the ribozyme technologies in a single construct. Smaller regions of homology are required for ribozyme catalysis, therefore this can promote the repression of different members of a large gene family if the cleavage sites are conserved.

NEMO and CYLD Molecules

Generally, the screening assays described herein involve a NEMO and a CYLD protein, or nucleic acid encoding such. NEMO and the NEMO-binding molecule CYLD and nucleic acids encoding these proteins are known in the art. The nucleotide sequence of a DNA encoding NEMO, and amino acid sequence encoded by this DNA, are set forth in SEQ ID NOs:1 and 2, respectively. The nucleotide sequence of a DNA encoding CYLD, and amino acid sequence encoded by this DNA, are set forth in SEQ ID NOs:3 and 4, respectively. However, it is understood that other NEMO and CYLD variants other than those shown in these examples may be used in the hereindisclosed assays, including other NEMO and CYLD molecules known in the art, or variants having similar properties.

Sequence variants of native NEMO and CYLD polypeptides are useful in the practice of the present invention in any instance where the native NEMO or CYLD polypeptide is utilized, provided that the variant possesses any biological activity required for the assay. Generally for these assays, suitable NEMO variants will bind CYLD. Mutations present in such variants may include, for example, substitutions, deletions, and insertions of amino acids. Allelic forms or mutated forms of NEMO and CYLD can be obtained for use in these assays by using a variety of techniques known in the art, including, for example, site-directed mutagenesis, oligonucleotide-directed mutagenesis, and so on.

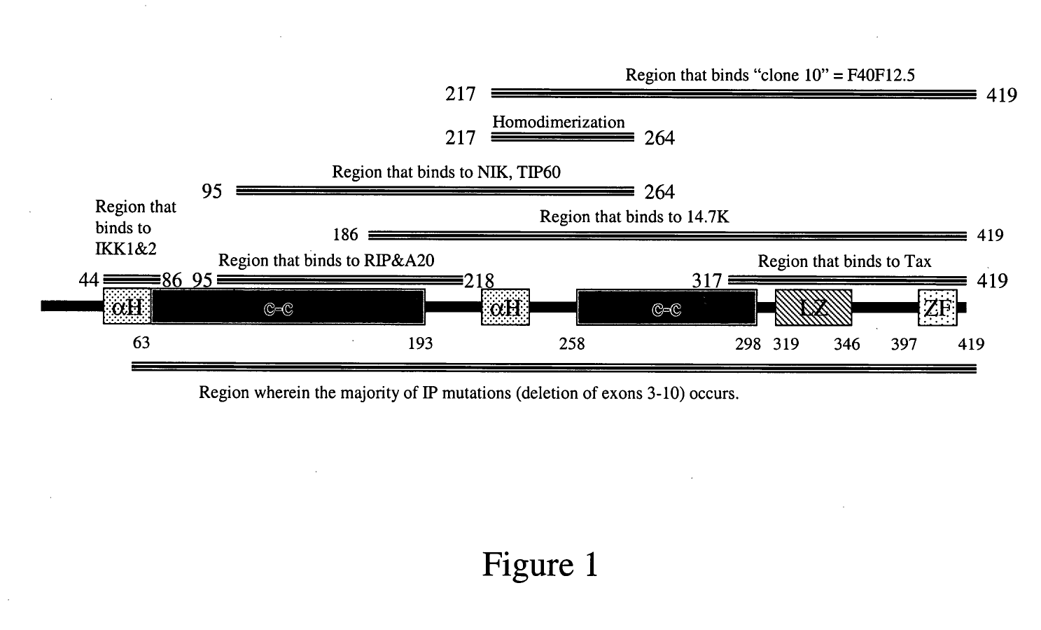

Also useful in the inventive methods are fragments of NEMO and/or CYLD. Particularly useful fragments of NEMO include the region from about amino acid 300 to 419, comprising a leucine zipper and zinc finger domain, and the region from about amino acid 387 to 419, comprising the zinc finger domain (see FIG. 1); additional fragments thereof that bind CYLD can be identified as described herein, and will also be useful in the present methods. Such fragments include those that are truncated by about five to ten amino acids (i.e., fragments from x to y, wherein x is selected from the group consisting of 386, 385, 384, 3843, 382, 381, 380 379, 378 and 377, and y is selected from the group consisting of 409, 410, 411, 412, 413, 414, 415, 416, 417, 418 and 419, and in particular, 418 and 419), and those having an N-terminus between amino acid 300 and 387 (i.e., fragments from x to y, wherein x is an integer between 300 and 387, and y is selected from the group consisting of 409, 410, 411, 412, 413, 414, 415, 416, 417, 418 and 419). Particularly useful fragments of CYLD include those that are capable of binding NEMO.

Sequence variants of NEMO and CYLD polypeptides that are not capable of binding their native binding partner may also useful in any of the assays described herein. In one embodiment, such sequence variants are useful as controls for an assay. In another embodiment, sequence variants that do not bind their respective binding partners will be useful in screening for molecules that facilitate the binding of NEMO and CYLD despite the inability of the sequence variants to bind in the absence of the facilitating molecule. Such facilitating molecules will be useful in treating disease conditions characterized by an inability of NEMO to bind to CYLD (i.e., X-linked hyper IgM with ectodermal dysplasia). Useful sequence variants can be obtained as described herein.

NEMO and/or CYLD peptides that are useful in the inventive methods (including fragments such as those mentioned previously) may be expressed as fusion proteins with tag peptides that facilitate detection and/or purification. Such peptides include, for example, poly-His or the antigenic identification peptides described in U.S. Pat. No. 5,011,912 and in Hopp et al., Bio/Technology 6:1204, 1988. Additional, useful tag proteins include green fluorescent protein (GFP; Chalfie et al., Science 263:802, 1994), an N-terminal peptide that contains recognition sites for a monoclonal antibody, a specific endopeptidase, and a site-specific protein kinase (PKA; Blanar and Rutter, Science 256:1014, 1992), birA (Altman et al., Science 274:94, 1996) and glutathione S transferase (GST: Smith and Johnson, Gene 67:31, 1988).

One such tag peptide is the FLAG™ peptide, which is highly antigenic and provides an epitope reversibly bound by a specific monoclonal antibody, enabling rapid assay and facile purification of expressed recombinant protein. A murine hybridoma designated 4E11 produces a monoclonal antibody that binds the FLAG™ peptide in the presence of certain divalent metal cations, as described in U.S. Pat. No. 5,011,912, hereby incorporated by reference. The 4E11 hybridoma cell line has been deposited with the American Type Culture Collection under accession no. HB 9259. Monoclonal antibodies that bind the FLAG™ peptide are available from Eastman Kodak Co., Scientific Imaging Systems Division, New Haven, Conn.

Another useful tag peptide is the GST peptide, which binds glutathione, also facilitating purification of expressed recombinant protein. Recombinant protein can be purified by affinity chromatography using a suitable chromatography matrix to which has been attached glutathione, as described in Smith and Johnson, supra, hereby incorporated by reference. Suitable chromatography matrixes include Glutathione-Agarose beads (Pharmacia, Uppsala, Sweden). Recombinant protein can be eluted with an excess of glutathione. Fragments of NEMO comprising the zinc finger domain (as described above) and a GST peptide are preferred.

The proteins useful in the practice of the present invention typically have an amino acid sequence that is at least 80% identical, or at least 85% identical, or preferably at least 90% identical to all or a portion of the corresponding native protein as set forth in SEQ ID NOS:2 or 4. Percent identity is defined as the percentage of the amino acid residues set forth in SEQ ID NOS:2, or 4, that are identical with part or all of another protein sequence (which may be a portion of a larger protein sequence) after aligning the sequences and introducing gaps, if necessary, to achieve the maximum percent identity. For comparing amino acid sequences of unequal length, the percent identity is calculated based on the smaller of the two sequences.

Preferably, the comparison is done using a computer program. An exemplary, preferred computer program is the Genetics Computer Group (GCG; Madison, Wis.) Wisconsin package version 10.0 program, ‘GAP.’ The preferred default parameters for the ‘GAP’ program includes: (1) The GCG implementation of the previously stated comparison matrixes for nucleotides and amino acids; (2) a penalty of 30 for each gap and an additional penalty of 1 for each symbol in each gap for amino acid sequences, or penalty of 50 for each gap and an additional penalty of 3 for each symbol in each gap for nucleotide sequences; (3) no penalty for end gaps; and (4) no maximum penalty for long gaps. Another program useful for determining percent identify is the BESTFIT program, also available from the University of Wisconsin as part of the GCG computer package. Default parameters for using the BESTFIT program are the same as those described above for using the GAP program.

Screening Assays for NEMO and CYLD:

Specific screening methods are known in the art and along with integrated robotic systems and collections of chemical compounds/natural products are extensively incorporated in high throughput screening so that large numbers of test compounds can be tested for antagonist or agonist activity within a short amount of time. These methods include homogeneous assay formats such as fluorescence resonance energy transfer, fluorescence polarization, time-resolved fluorescence resonance energy transfer, scintillation proximity assays, reporter gene assays, fluorescence quenched enzyme substrate, chromogenic enzyme substrate and electrochemiluminescence, as well as more traditional heterogeneous assay formats such as enzyme-linked immunosorbant assays (ELISA) or radioimmunoassays. Homogeneous assays are preferred. Also comprehended herein are cell-based assays, for example those utilizing reporter genes, as well as functional assays that analyze the effect of an antagonist or agonist on biological function(s) (for example, secretion of cytokines or immunoglobulin class switching).

Moreover, combinations of screening assays can be used to find molecules that regulate the biological activity of NEMO and/or CYLD. Molecules that regulate the biological activity of a polypeptide may be useful as agonists or antagonists of the peptide. In using combinations of various assays, it is usually first determined whether a candidate molecule binds to a polypeptide by using an assay that is amenable to high throughput screening. Binding candidate molecules identified in this manner are then added to a biological assay to determine biological effects. Molecules that bind and that have an agonistic or antagonistic effect on biologic activity will be useful in treating or preventing disease or conditions with which the polypeptide(s) are implicated.

Generally, an antagonist will inhibit the activity by at least 30%; more preferably, antagonists will inhibit activity by at least 50%, most preferably by at least 90%. Similarly, an agonist will enhance the activity by at least 20%; more preferably, agonists will enhance activity by at least 30%, most preferably by at least 50%. Those of skill in the art will recognize that agonists and/or antagonists with different levels of agonism or antagonism respectively may be useful for different applications (i.e., for treatment of different disease states).

Homogeneous assays are mix-and-read style assays that are very amenable to robotic application, whereas heterogeneous assays require separation of free from bound analyte by more complex unit operations such as filtration, centrifugation or washing. These assays are utilized to detect a wide variety of specific biomolecular interactions (including protein-protein, receptor-ligand, enzyme-substrate, and so on), and the inhibition thereof by small organic molecules. These assay methods and techniques are well known in the art (see, e.g., High Throughput Screening: The Discovery of Bioactive Substances, John P. Devlin (ed.), Marcel Dekker, New York, 1997 ISBN: 0-8247-0067-8). The screening assays of the present invention are amenable to high throughput screening of chemical libraries and are suitable for the identification of small molecule drug candidates, antibodies, peptides, and other antagonists and/or agonists, natural or synthetic.

One such assay is based on fluorescence resonance energy transfer (FRET; for example, HTRF®, Packard BioScience Company, Meriden, Conn.; LANCE™, PerkinElmer LifeSciences, Wallac Oy., Turku, Finland) between two fluorescent labels, an energy donating long-lived chelate label and a short-lived organic acceptor. The energy transfer occurs when the two labels are brought in close proximity via the molecular interaction between NEMO and CLD. In a FRET assay for detecting inhibition of the binding of NEMO and CYLD, europium chelate or cryptate labeled NEMO or CYLD serves as an energy donor and streptavidin-labeled allophycocyanin (APC) bound to the appropriate binding partner (i.e., CYLD if NEMO is labeled, or NEMO if CYLD is labeled) serves as an energy acceptor. Once NEMO binds CYLD, the donor and acceptor molecules are brought in close proximity, and energy transfer occurs, generating a fluorescent signal at 665 nm. Antagonists of the interaction of NEMO and CYLD will thus inhibit the fluorescent signal, whereas agonists of this interaction would enhance it.

Another useful assay is a bioluminescence resonance energy transfer, or BRET, assay, substantially as described in Xu et al., Proc. Natl. Acad. Sci. USA 96:151 (1999). Similar to a FRET assay, BRET is based on energy transfer from a bioluminescent donor to a fluorescent acceptor protein. However, a green fluorescent protein (GFP) is used as the acceptor molecule, eliminating the need for an excitation light source. Exemplary BRET assays include BRET and BRET² from Packard BioScience, Meriden, Conn.

DELFIA® (dissociated enhanced lanthanide fluoroimmunoassay; PerkinElmer LifeSciences, Wallac Oy., Turku, Finland) is a solid-phase assay based on time-resolved fluorometry analysis of lanthanide chelates (see, for example, U.S. Pat. No. 4,565,790 issued Jan. 21, 1986). For this type of assay, microwell plates are coated with a first protein (NEMO or CYLD). The binding partner (CYLD or NEMO, respectively) is conjugated to europium chelate or cryptate, and added to the plates. After suitable incubation, the plates are washed and a solution that dissociates europium ions from solid phase bound protein, into solution, to form highly fluorescent chelates with ligands present in the solution, after which the plates are read using a reader such as a VICTOR²™ (PerkinElmer LifeSciences, Wallac Oy., Turku, Finland) plate reader to detect emission at 615 nm).

Another assay that will be useful in the inventive methods is a FlashPlate® (Packard Instrument Company, IL)-based assay. This assay measures the ability of compounds to inhibit protein-protein interactions. FlashPlates® are coated with a first protein (either NEMO or CYLD), then washed to remove excess protein. For the assay, compounds to be tested are incubated with the second protein (CYLD, if the plates are coated with NEMO, or NEMO if plates are coated with CYLD) and I¹²⁵ labeled antibody against the second protein and added to the plates. After suitable incubation and washing, the amount of radioactivity bound is measured using a scintillation counter (such as a MicroBeta® counter; PerkinElmer LifeSciences, Wallac Oy., Turku, Finland).

The AlphaScreen™ assay (Packard Instrument Company, Meriden, Conn.). AlphaScreen™ technology is an “Amplified Luminescent Proximity Homogeneous Assay” method utilizing latex microbeads (250 nm diameter) containing a photosensitizer (donor beads), or chemiluminescent groups and fluorescent acceptor molecules (acceptor beads). Upon illumination with laser light at 680 nm, the photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity to the donor bead (i.e., by virtue of the interaction of NEMO and CYLD), the singlet-state oxygen molecules reacts with chemiluminescent groups in the acceptor beads, which immediately transfer energy to fluorescent acceptors in the same bead. These fluorescent acceptors shift the emission wavelength to 520-620 nm, resulting in a detectable signal. Antagonists of the interaction of NEMO and CYLD will thus inhibit the shift in emission wavelength, whereas agonists of this interaction would enhance it.

One embodiment of a method for identifying molecules which inhibit or antagonize CD40-mediated signaling involves adding a candidate molecule to a medium which contains cells that express NEMO and CYLD; changing the conditions of said medium so that, but for the presence of the candidate molecule, NEMO would be bound to CYLD, and observing the binding and stimulation or inhibition of a functional response. The activity of the cells that were contacted with the candidate molecule may then be compared with the identical cells that were not contacted and antagonists and agonists of the polypeptides of the instant invention may be identified. The measurement of biological activity may be performed by a number of well-known methods such as measuring the amount of protein present (e.g. an ELISA) or of the protein's activity. A decrease in biological stimulation or activation would indicate an antagonist. An increase would indicate an agonist.

Computer Analysis of Assay Results

In one aspect of the invention, the assays of the invention are used to identify compounds that alter CD40 signaling activity. The benefits of integrated robotic systems used to analyze collections of chemical compounds/natural products in such assays, which preferably incorporate high-throughput screening methods, are most often realized by the use of sophisticated computer and statistical techniques to manage the resulting data. In one form, the information generated in the inventive screening assays is stored (or compiled) in electronic form, using a computerized database that allows information to be efficiently catalogued and retrieved. Such databases are comprised of records, usually one record for each compound, that includes information about the compound, such as chemical name, structure, source, activity in a binding assay, activity in a biological assay, etc.

The information may be entered into the database manually, that is by a user entering data through a user interface (i.e., keyboard, touchpad, etc.), or it may be entered electronically as in when a robotic system for analysis of compounds generates electronic results that are transferred to another computer system (often referred to as uploading). Such information is usually stored in a discrete area of the record referred to as a field. Additionally, the information, preferably in the form of a database, may be stored permanently or temporarily on various forms of storage media, including paper, the brains of living organisms, compact disks, floppy disks, magnetic tapes, optical tapes, hard drives, computer system memory units, and the like.

The database may be stand-alone, or the records therein may be related to other databases (a relational database). Examples of other databases include publicly available, well-known databases such as GenBank for peptides and nucleic acids (and associated databases maintained by the National Center for Biotechnology Information or NCBI), and the databases available through www.chemfinder.com or The Dialog Corporation (Cary, N.C.) for chemical compounds.

A user will be able to search the database according to the information recorded (selecting records that have a particular value in a selected field, for example, searching for all compounds that inhibited a binding assay by at least about 30%); accordingly, another aspect of the invention is a method of using a computer system to catalog and store information about various chemical compounds. The ability to store and retrieve such information in computerized form allows those of ordinary skill in the art to select compounds for additional testing, including additional analysis of binding ability, biological testing, and testing in animal models or clinical trials of pharmaceutical agents in humans. Moreover, in addition to storing and cataloging information, the database can be used to provide a report, either in electronic form or in the form of a printout, that will facilitate further analysis of selected compounds.

One embodiment of the invention comprises a computing environment; an input device, connected to the computing environment, to receive information from the user; an output device, connected to the computing environment, to provide information to the user; and a plurality of algorithms selectively executed based on at least a portion of the received information, wherein any one of these algorithms analyzes at least a portion of the received information and generates output information, and preferably wherein the output information is communicated via the output device. The computing environment preferably further comprises a communications network; a server connected to the network; and a client connected to the network, wherein the client is part of a client-server architecture and typically is an application that runs on a personal computer or workstation and relies on a server to perform some operations (see Nath, 1995, The Guide To SQL Server, 2nd ed., Addison-Wesley Publishing Co.).

The computing environment of the present invention is advantageously implemented using any multipurpose computer system including those generally referred to as personal computers and mini-computers. Such a computer system will include means for processing input information such as at least one central processor, for example an Intel® processor (including Pentium® Pentium® II, Celeron™, Pentium® II3, Pentium® 4 or the like), or Motorola processor (for example, a PowerPC G3 or PowerPC G4 microprocessor capable of running at speeds up to 533 MHz or higher); a storage device, such as a hard disk, for storing information related to CD40, NEMO and/or CYLD polypeptides and/or compounds that alter the binding of NEMO and CYLD (or signaling through CD40); and means for receiving input information. Those of skill in the art recognize that computer technology is changing at a rapid rate; accordingly, new, improved versions of processors are comprehended herein.

The processor, which comprises and/or accesses memory units of the computer system, is programmed to perform analyses of information related to the CD40, NEMO and/or CYLD polypeptides and/or compounds that modulate the binding of NEMO and CYLD (or signaling through CD40). This programming may be permanent, as in the case where the processor is a dedicated PROM (programmable read-only memory) or EEPROM (electrically erasable programmable read-only memory), or it may be transient in which case the programming instructions are loaded from the storage device or from a floppy diskette or other transportable computer-readable media. The computing environment further preferably comprises a user interface such as a Unix/X-Window interface, a Microsoft Windows interface, or a Macintosh operating system interface.

Preferably, the computing environment further includes an optical disk for storing data, a printer for providing a hard copy of the data, and a monitor or video display unit to facilitate user input of information and to display both input and output information. The output information may be output from the processor within the computer system in print form using a printer; on a video display unit; or via a communications link or network to another processor or client application.

The following examples are intended to illustrate various embodiments of the invention, and should not be construed to limit the invention.

### EXAMPLE 1

This example describes a gene promoter/reporter system based on the human Interleukin-8 (IL-8) promoter used to analyze the activation of gene transcription in vivo. Other NF-kappaB-responsive promoters besides IL-8 could be used including a minimal promoter element comprising NF-kappaB consensus binding sites. The induction of human IL-8 gene transcription by the cytokines Interleukin-1 (IL-1) or tumor necrosis factor-alpha (TNF-α) is known to be dependent upon intact NF-kappaB and NF-IL-6 transcription factor binding sites. Fusion of the cytokine-responsive IL-8 promoter with a cDNA encoding the murine IL-4 receptor (mIL-4R) allows measurement of promoter activation by detection of the heterologous reporter protein (mIL-4R) on the cell surface of transfected cells. Other detectable moieties may be used, including any protein or peptide that is detectable by a selected assay and is not present on or in the cells under other conditions (for example, luciferase or human IL-2 receptor).

Human kidney epithelial cells (293/EBNA) are transfected (via the DEAE/DEXTRAN method) with a plasmid encoding the reporter/promoter construct (referred to as pIL-8rep), and cultured under conditions promoting viability. If CD40 activation is necessary for NEMO/CYLD association, 293 cells are transfected with plasmids encoding CD40. Alternatively, the NF-kappaB responsive promoter is introduced (by transfection) into cells that express endogenous CD40 (for example, 70Z/3, WEHI-231, or RAW 264.7 cell-lines). Stimulation of CD40 may be accomplished via addition of soluble CD40L, agonistic antibodies to CD40; or by cotransfection with the transmembrane form of CD40L; use of soluble CD40L is preferred.

The transfected cells are contacted with compounds to be tested. If a compound binds NEMO or CYLD, and inhibits the interaction of these two proteins, NF-kappaB will remain sequestered and unable to activate the IL-8 promoter. As a result, there will be no mIL-4R present on the surface of cells that have been contacted with an effective antagonist. The presence or absence of the mIL-4 receptor is detected by a radioimmunoassay (RIA) or other suitable assay.

### EXAMPLE 2

This example illustrates the association of NEMO with CYLD. Interaction of NEMO with CYLD is demonstrated by co-immunoprecipitation assays essentially as described by Hsu et al. (Cell 84:299; 1996). Briefly, 293/EBNA cells are co-transfected with plasmids that direct the synthesis of NEMO and epitope-tagged CYLD (or CYLD and epitope-tagged NEMO). Other cells may also be used, including cells that constitutively express NEMO and/or CYLD (for example, 70Z/3 cells). Two days after transfection, cells are lysed (for example, in a buffer containing 0.5% NP-40 and labeled (for example, with biotin-ester). NEMO and proteins associated with NEMO are immunoprecipitated with anti-NEMO (or anti-CYLD), washed extensively, resolved by electrophoretic separation on a 6-10% SDS polyacrylamide gel and electrophoretically transferred to a nitrocellulose membrane for Western blotting. The association of NEMO with CYLD is visualized by probing the membrane with an antibody that specifically recognizes the tag.

### EXAMPLE 3

This example describes a yeast two-hybrid screening assay that is useful in screening compounds for the ability to modulate the binding of NEMO and CYLD. Yeast comprising (1) an expression cassette encoding a GAL4 DNA binding domain (or GAL4 activator domain) fused to a binding fragment of NEMO capable of binding to a CYLD polypeptide, (2) an expression cassette encoding a GAL4 DNA activator domain (or GAL4 binding domain, respectively) fused to a binding fragment of CYLD capable of binding to a NEMO polypeptide, and (3) a reporter gene (e.g., beta-galactosidase) comprising a cis-linked GAL4 transcriptional response element can be used for agent screening. Such yeast are incubated with a test agent or the appropriate control(s) under conditions promoting expression of the reporter gene in the absence of an inhibitor, and expression of the reporter is determined. The capacity of the agent to modulate expression of the reporter gene as compared to a control culture identifies the agent as a candidate modulatory agent.

### EXAMPLE 4

This example illustrates the preparation of monoclonal antibodies against NEMO or CYLD. Preparations of purified NEMO or CYLD, for example, or transfixed cells expressing high levels of NEMO or CYLD, are employed to generate monoclonal antibodies against NEMO or CYLD using conventional techniques, such as those disclosed in U.S. Pat. No. 4,411,993, incorporated herein by reference. DNA encoding NEMO or CYLD can also be used as an immunogen, for example, as reviewed by Pardoll and Beckerleg in Immunity 3: 165, 1995. Such antibodies are likely to be useful in interfering with NEMO/CYLD binding (antagonistic or blocking antibodies), as components of diagnostic or research assays for NEMO or CYLD activity, or in affinity purification of NEMO or CYLD.

To immunize rodents, NEMO or CYLD immunogen is emulsified in an adjuvant (such as complete or incomplete Freund's adjuvant, alum, or another adjuvant, such as Ribi adjuvant R700 (Ribi, Hamilton, Mont.), and injected in amounts ranging from 10-100 μg subcutaneously into a selected rodent, for example, BALB/c mice or Lewis rats. DNA may be given intradermally (Raz et al., Proc. Natl. Acad. Sci. USA 91: 9519, 1994) or intramuscularly (Wang et al., Proc. Natl. Acad. Sci. USA 90: 4156, 1993); saline has been found to be a suitable diluent for DNA-based antigens. Ten days to three weeks days later, the immunized animals are boosted with additional immunogen and periodically boosted thereafter on a weekly, biweekly or every third week immunization schedule.

Serum samples are periodically taken by retro-orbital bleeding or tail-tip excision for testing by dot-blot assay (antibody sandwich), ELISA (enzyme-linked immunosorbent assay), immunoprecipitation, or other suitable assays, including FACS analysis. Following detection of an appropriate antibody titer, positive animals are given an intravenous injection of antigen in saline. Three to four days later, the animals are sacrificed, splenocytes harvested, and fused to a murine myeloma cell line (e.g., NS1 or preferably Ag 8.653 [ATCC CRL 1580]). Hybridoma cell lines generated by this procedure are plated in multiple microtiter plates in a selective medium (for example, one containing hypoxanthine, aminopterin, and thymidine, or HAT) to inhibit proliferation of non-fused cells, myeloma-myeloma hybrids, and splenocyte-splenocyte hybrids.

Hybridoma clones thus generated can be screened by ELISA for reactivity with NEMO or CYLD, for example, by adaptations of the techniques disclosed by Engvall et al., Immunochem. 8: 871 (1971) and in U.S. Pat. No. 4,703,004. A preferred screening technique is the antibody capture technique described by Beckman et al., J. Immunol. 144: 4212 (1990). Positive clones are then injected into the peritoneal cavities of syngeneic rodents to produce ascites containing high concentrations (>1 mg/ml) of monoclonal antibody. The resulting monoclonal antibody can be purified by ammonium sulfate precipitation followed by gel exclusion chromatography. Alternatively, affinity chromatography based upon binding of antibody to protein A or protein G can also be used, as can affinity chromatography based upon binding to NEMO or CYLD protein.

Techniques described for the production of single chain antibodies (U.S. Pat. No. 4,946,778; Bird, Science 242:423, 1988; Huston et al., Proc. Natl. Acad. Sci. USA 85:5879, 1988; and Ward et al., Nature 334:544, 1989) can also be adapted to produce single chain antibodies against NEMO or CYLD. Single chain antibodies are formed by linking the heavy and light chain fragments of the Fv region via an amino acid bridge, resulting in a single chain polypeptide. Such single chain antibodies can be useful intracellularly (i.e., as intrabodies), for example as described by Marasco et al. (J. Immunol. Methods 231:223-238, 1999) for genetic therapy in HIV infection. Intrabodies that bind NEMO or CYLD and inhibit the interaction thereof will be useful in downregulating specific CD40-signaling response.

### EXAMPLE 5

In accordance with the present invention, a series of oligonucleotides are designed to target different regions of the NEMO or CYLD mRNA molecule, using the nucleotide sequence of SEQ ID Nos:1 or 3, respectively, as the basis for the design of the oligonucleotides. The oligonucleotides are selected to be approximately 10, 12, 15, 18, or more preferably 20 nucleotide residues in length, and to have a predicted hybridization temperature that is at least 37 degrees C. Preferably, the oligonucleotides are selected so that some will hybridize toward the 5′ region of the mRNA molecule, others will hybridize to the coding region, and still others will hybridize to the 3′ region of the mRNA molecule.

The oligonucleotides may be oligodeoxynucleotides, with phosphorothioate backbones (internucleoside linkages) throughout, or may have a variety of different types of internucleoside linkages. Generally, methods for the preparation, purification, and use of a variety of chemically modified oligonucleotides are described in U.S. Pat. No. 5,948,680. As specific examples, the following types of nucleoside phosphoramidites may be used in oligonucleotide synthesis: deoxy and 2′-alkoxy amidites; 2′-fluoro amidites such as 2′-fluorodeoxyadenosine amidites, 2′-fluorodeoxyguanosine, 2′-fluorouridine, and 2′-fluorodeoxycytidine; 2′-O-(2-methoxyethyl)-modified amidites such as 2,2′-anhydro[1-(beta-D-arabino-furanosyl)-5-methyluridine], 2′-O-methoxyethyl-5-methyluridine, 2′-O-methoxyethyl-5′-O-dimethoxytrityl-5-methyluridine, 3′-O-acetyl-2′-O-methoxy-ethyl-5′-O-dimethoxy-trityl-5-methyluridine, 3′-O-acetyl-2′-O-methoxyethyl-5′-O-dimethoxytrityl-5-methyl-4-triazoleuridine, 2′-O-methoxyethyl-5′-O-dimethoxy-trityl-5-methylcytidine, N4-benzoyl-2′-O-methoxyethyl-5′-O-dimethoxytrityl-5-methyl-cytidine, and N4-benzoyl-2′-O-methoxyethyl-5′-O-di-methoxytrityl-5-methylcytidine-3′-amidite; 2′-O-(aminooxyethyl) nucleoside amidites and 2′-O-(dimethylaminooxyethyl) nucleoside amidites such as 2′-(dimethylaminooxyethoxy) nucleoside amidites, 5′-O-tert-butyldiphenylsilyl-O²-2′-anhydro-5-methyluridine, 5′-O-tert-butyl-diphenylsilyl-2′-O-(2-hydroxyethyl)-5-methyluridine, 2′-O-([2-phthalimidoxy)ethyl]-5′-t-butyl-diphenyl-silyl-5-methyluridine, 5′-O-tert-butyl-diphenylsilyl-2′-O-[(2-formadoximinooxy)ethyl]-5-methyl-uridine, 5′-O-tert-butyl-diphenylsilyl-2′-O-[N,N-dimethyl-aminooxyethyl]-5-methyluridine, 2′-O-(dimethyl-aminooxy-ethyl)-5-methyluridine, 5′-O-DMT-2′-O-(dimethylamino-oxyethyl)-5-methyluridine, and 5′-O-DMT-2′-O-(2-N,N-dimethylaminooxyethyl)-5-methyluridine-3′-[(2-cyanoethyl)-N,N-diisopropyl-phosphor-amidite]; and 2′-(amino-oxyethoxy) nucleoside amidites such as N2-isobutyryl-6-O-diphenyl-carbamoyl-2′-O-(2-ethylacetyl)-5′-O-(4,4′-dimethoxytrityl)-guanosine-3′-[(2-cyanoethyl)-N,N-diiso-propylphosphoramidite].

Modified oligonucleosides may also be used in oligonucleotide synthesis, for example methylenemethylimino-linked oligonucleosides, also called MMI-linked oligonucleosides; methylene-dimethylhydrazo-linked oligonucleosides, also called MDH-linked oligonucleosides; methylene-carbonylamino-linked oligonucleosides, also called amide-3-linked oligonucleosides; and methylene-aminocarbonyl-linked oligonucleosides, also called amide-4-linked oligonucleosides, as well as mixed backbone compounds having, for instance, alternating NMI and P═O or P═S linkages, which are prepared as described in U.S. Pat. Nos. 5,378,825, 5,386,023, 5,489,677, 5,602,240 and 5,610,289. Formacetal- and thioformacetal-linked oligonucleosides may also be used and are prepared as described in U.S. Pat. Nos. 5,264,562 and 5,264,564; and ethylene oxide linked oligonucleosides may also be used and are prepared as described in U.S. Pat. No. 5,223,618. Peptide nucleic acids (PNAs) may be used as in the same manner as the oligonucleotides described above, and are prepared in accordance with any of the various procedures referred to in Peptide Nucleic Acids (PNA): Synthesis, Properties and Potential Applications, Bioorganic & Medicinal Chemistry, 1996, 4, 5-23; and U.S. Pat. Nos. 5,539,082, 5,700,922, and 5,719,262.

Chimeric oligonucleotides, oligonucleosides, or mixed oligonucleotides-oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”. Some examples of different types of chimeric oligonucleotides are: [2′-O-Me]—[2′-deoxy]—[2′-O-Me] chimeric phosphorothioate oligonucleotides, [2′-O-(2-methoxyethyl)]—[2′-deoxy]—[2′-O-(methoxyethyl)] chimeric phosphorothioate oligonucleotides, and [2′-O-(2-methoxy-ethyl)phosphodiester]—[2′-deoxyphosphoro-thioate]—[2′-O-(2-methoxyethyl)-phosphodiester] chimeric oligonucleo-tides, all of which may be prepared according to U.S. Pat. No. 5,948,680. In one preferred embodiment, chimeric oligonucleotides (“gapmers”) 18 nucleotides in length are utilized, composed of a central “gap” region consisting of ten 2′-deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. Other chimeric oligonucleotides, chimeric oligonucleosides, and mixed chimeric oligonucleotides/oligonucleosides are synthesized according to U.S. Pat. No. 5,623,065.

Oligonucleotides are preferably synthesized via solid phase P(III) phosphoramidite chemistry on an automated synthesizer capable of assembling 96 sequences simultaneously in a standard 96 well format. The concentration of oligonucleotide in each well is assessed by dilution of samples and UV absorption spectroscopy. The full-length integrity of the individual products is evaluated by capillary electrophoresis, and base and backbone composition is confirmed by mass analysis of the compounds utilizing electrospray-mass spectroscopy.

The effect of antisense compounds on target nucleic acid expression can be tested in any of a variety of cell types provided that the target nucleic acid is present at measurable levels. This can be routinely determined using, for example, PCR or Northern blot analysis. Cells are routinely maintained for up to 10 passages as recommended by the supplier. When cells reach 80% to 90% confluency, they are treated with oligonucleotide. For cells grown in 96-well plates, wells are washed once with 200 microliters OPTI-MEM-1 reduced-serum medium (Gibco BRL) and then treated with 130 microliters of OPTI-MEM-1 containing 3.75 g/mL LIPOFECTIN (Gibco BRL) and the desired oligonucleotide at a final concentration of 150 nM. After 4 hours of treatment, the medium is replaced with fresh medium. Cells are harvested 16 hours after oligonucleotide treatment. Preferably, the effect of several different oligonucleotides should be tested simultaneously, where the oligonucleotides hybridize to different portions of the target nucleic acid molecules, in order to identify the oligonucleotides producing the greatest degree of inhibition of expression of the target nucleic acid.

Antisense modulation of NEMO or CYLD nucleic acid expression can be assayed in a variety of ways known in the art. For example, NEMO or CYLD mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation and Northern blot analysis are taught in, for example, Ausubel, F. M. et al., Current Protocols in Molecular Biology, Volume 1, pp. 4.1.1-4.2.9 and 4.5.1-4.5.3, John Wiley & Sons, Inc., 1996.

Real-time quantitative (PCR) can be conveniently accomplished using the commercially available ABI PRISM 7700 Sequence Detection System, available from PE-Applied Biosystems, Foster City, Calif. and used according to manufacturer's instructions. This fluorescence detection system allows high-throughput quantitation of PCR products. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe.

When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples. Other methods of quantitative PCR analysis are also known in the art.

NEMO or CYLD protein levels can be quantitated in a variety of ways well known in the art, such as immunoprecipitation, Western blot analysis (immunoblotting), ELISA, or fluorescence-activated cell sorting (FACS). Antibodies directed to NEMO or CYLD polypeptides can be prepared via conventional antibody generation methods such as those described herein. Immunoprecipitation methods, Western blot (immunoblot) analysis, and enzyme-linked immunosorbent assays (ELISA) are standard in the art (see, for example, Ausubel, F. M. et al., Current Protocols in Molecular Biology, Volume 2, pp. 10.16.1-10.16.11, 10.8.1-10.8.21, and 11.2.1-11.2.22, John Wiley & Sons, Inc., 1991).

### EXAMPLE 6

This example describes a mammalian two-hybrid screening assay (substantially as described in Shioda et al., Proc. Natl. Acad. Sci. USA 97:5220, 2000) that is useful in screening compounds for the ability to modulate the binding of NEMO and CYLD. For this assay, CV-1/EBNA-1 monkey kidney epithelial cells expressing Epstein-Barr virus nuclear antigen 1 (EBNA-1) are stably transfected with a reporter plasmid for GAL4-dependent expression of the green fluorescent protein (GFP). Clones that express GFP when transfected transiently with transcriptional activators fused to GAL4 DNA-binding domain with minimal background GFP expression are identified.

Useful clones are then transfected stably with a model bait (for example, NEMO or CYLD) and prey (CYLD or NEMO, respectively); under conditions in which both NEMO and CYLD are expressed and allowed to bind to each other, the cells will express GFP and can be readily identified by green fluorescence in cell culture. Such cells are incubated with a test agent or the appropriate control(s) and the capacity of the agent to modulate expression of GFP as compared to a control culture is deterrmined. Those of skill in the art will be able to select other reporter genes as desired.

### EXAMPLE 7

In this assay, test molecules capable of modulating an activity associated with CD40 signaling are identified by assessing the ability of B cells to undergo immunoglobulin class switching, and/or the ability of peripheral blood mononuclear cells (PBMCs) to synthesize IL-12 and/or TNF-alpha. Human peripheral blood mononuclear cells (PBMC) are isolated from peripheral blood from normal volunteers by density gradient centrifugation over Histopaque® (Sigma, St. Louis, Mo.). T cell-depleted preparations of cells (E⁻) are obtained by removing T cells by resetting with 2-aminoethylisothiouronium bromide-treated SRBC (sheep red blood cells) and further density gradient centrifugation over Histopaque®. Alternatively, B cells are selectively isolated using anti CD19-coated magnetic beads (Dynal, Lake Success, N.Y.).

B cell assays are conducted in RPMI media with added 10% heat-inactivated fetal bovine serum (FBS) at 37° C. in a 10% CO₂ atmosphere. Cells (approximately 1×10⁶ cells per well for observation of markers; approximately 1×10⁵ cells per well for detection of immunoglobulin in supernatant fluid) are cultured in the presence of 2.5 micrograms/ml of soluble, trimeric CD40L (described in the Armitage patents), for seven to eight days. Immunoglobulin concentrations (IgG, IgA) in the supernatant fluid are determined by specific ELISA; cells are monitored for the expression of CD54 by flow cytometry.

PBMC assays are conducted in RPMI 1640 complete medium. Approximately 2×10⁶ cells per well are cultured in the presence of 2.5 micrograms/ml of soluble, trimeric CD40L (described in the Armitage patents), for 36 to 48 hours. Concentration of IL-12 and/or TNF-alpha in the supernatant fluid are determined by specific ELISA; cells are monitored for the expression of molecules indicative of activation by flow cytometry.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method for determining an effect of a test compound on binding of Nuclear factor-kappaB Essential Modulator (NEMO) polypeptide to a polypeptide encoded by a putative tumor suppressor gene associated with familial cylindromatosis (CYLD), wherein the NEMO polypeptide is selected from the group consisting of:
(a) a NEMO polypeptide comprising amino acids 300 through 419 of SEQ ID NO:2;
(b) a fragment of a NEMO polypeptide comprising amino acids x through y of SEQ ID NO:2, wherein x is selected from the group consisting of 386, 385, 384, 383, 382, 381, 380, 379, 378 and 377, and y is selected from the group consisting of 409, 410, 411, 412, 413, 414, 415, 416, 417, 418 and 419, and
(c) a fragment of a NEMO polypeptide comprising amino acids x through y of SEQ ID NO:2, wherein x is an integer between 300 and 387, and y is selected from the group consisting of 409, 410, 411, 412, 413, 414, 415, 416, 417, 418 and 419;
and the CYLD polypeptide comprises amino acids 1 through 956 of SEQ ID NO:4, and further wherein the NEMO polypeptide and the CYLD polypeptide are capable of binding to each other, the method comprising the steps of:
(i) mixing the test compound with the NEMO polypeptide and the CYLD polypeptide: and
(ii) determining the effect of the test compound on the binding of the NEMO polypeptide to the CYLD polypeptide by comparing the binding of the NEMO polypeptide to the CYLD polypeptide in the presence of the test compound to the binding of the NEMO polypeptide to the CYLD polypeptide in the absence of the test compound, wherein when the amount of binding of the NEMO polypeptide to the CYLD polypeptide in the presence of the test compound is less than the binding of the NEMO polypeptide to the CYLD polypeptide in the absence of the test compound, the test compound inhibits the binding of NEMO and CYLD.

2. The method of claim 1, wherein the NEMO polypeptide is selected from the group consisting of:
(a) a NEMO polypeptide comprising amino acids 300 through 419 of SEQ ID NO:2; and
(b) a NEMO polypeptide comprising amino acids 387 through 419 of SEQ ID NO:2.

3. The method of claim 1, wherein the NEMO polypeptide is selected from the group consisting of:
(a) a NEMO polypeptide consisting essentially of amino acids 300 through 419 of SEQ ID NO:2;
(b) a fragment of a NEMO polypeptide consisting essentially of amino acids x through y of SEQ ID NO:2, wherein x is selected from the group consisting of 386, 385, 384, 383, 382, 381, 380, 379, 378 and 377, and y is selected from the group consisting of 409, 410, 411, 412, 413, 414, 415, 416, 417, 418 and 419; and
(c) a fragment of a NEMO polypeptide consisting essentially of amino acids x through y of SEQ ID NO:2, wherein x is an integer between 300 and 387, and y is selected from the group consisting of 409, 410, 411, 412, 413, 414, 415, 416, 417, 418 and 419.

4. A method for producing information comprising the identity of a compound that alters one or more biological activities of CD40, the method comprising identifying one or more compounds that alter NEMO/CYLD binding activity.

5. The method of claim 4 wherein the compound decreases the binding of NEMO to CYLD.

6. The method of claim 4 wherein the compound increases the binding of NEMO to CYLD.

7. Information produced according to the method of claim 4, said information comprising the identity of a compound that alters the binding of NEMO to CYLD.

8. A database comprising the information of claim 7.

9. A computer system comprising a database containing records pertaining to a plurality of compounds, wherein the records comprise results of an assay according to claim 1, and a user interface allowing a user to access information regarding the plurality of compounds.

10. A computer system comprising a database containing records pertaining to a plurality of compounds, wherein the records comprise results of an assay according to claim 2, and a user interface allowing a user to access information regarding the plurality of compounds.

11. A computer system for storing and retrieving data on a plurality of compounds, the computer system comprising:
(a) input means for entering data for the compounds into a storage medium;
a processor for creating an individual record for each compound, the processor assigning specific identifying values for each compound;
(b) means for selecting one or more of the records based on results in an assay according to claim 1; and
(c) means for transmitting information in the record or records to an output device to produce a report.

12. A method of using the computer system of claim 11 to select one or more compounds for testing from a plurality of compounds having records stored in a database, the method comprising: displaying a list of said records or a field for entering information identifying one or more of said records; and selecting one or more of the records from the list or the record or records identified by entering information in the field.

13. A method of operating a computer system for analyzing compounds that modulate the interaction of NEMO and CYLD, the method comprising:
(a) entering data relating to a plurality of compounds into a storage medium;
processing the data to create an individual record for each compound;
(b) testing compounds for the ability to modulate binding of NEMO to CYLD; and
(c) communicating results from the testing into the storage medium such that results for each compound are associated with the individual record for that compound.

14. A database comprising records generated according to the method of claim 13.

15. A method of selecting compounds that alter one or more biological activities of CD40, comprising compiling a database according to claim 14, analyzing the testing results, and selecting one or more compounds.

16. A method for increasing one or more biological activities of CD40 comprising providing at least one compound selected according to the method of claim 15.

17. A method for decreasing one or more biological activities of CD40 comprising providing at least one compound selected according to the method of claim 15.
